(19) United States
(12) Patent Application Publication
(10) Pub. No.: US 2009/0028849 A1
(43) Pub. Date: Jan. 29, 2009

(54) AGENTS AND METHODS FOR INHIBITION OF AIRWAY HYPERRESPONSIVENESS

(76) Inventors: Rebecca L. O'Brien, Denver, CO (US); Willi K. Born, Denver, CO (US); Niyun Jin, Denver, CO (US); Christina L. Roark, Englewood, CO (US)

Correspondence Address: SHERIDAN ROSS PC, 1560 BROADWAY, SUITE 1200, DENVER, CO, 80202, US

(21) Appl. No.: 12/178,923

(22) Filed: Jul. 24, 2008

Related U.S. Application Data
(60) Provisional application No. 60/951,589, filed on Jul. 24, 2007.

(51) Int. Cl. A61K 39/395; A61K 38/17; A61P 11/00

(52) U.S. Cl. 424/130.1; 514/12; 424/93.71

(57) ABSTRACT

The present invention provides methods and compositions for inhibition of Airway Hyperresponsiveness (“AHR”) by deleting, inactivating or inhibiting a subset of innate γδ T cells, alone or in conjunction with the inhibition of Natural Killer T (“NKT”) cells. The methods comprise selective leukophoresis to remove the γδ T cells from the individual's blood or administration of an agent that selectively targets and inhibits or inactivates the γδ T cells. The methods of the present invention can be used to treat AHR in a variety of different ailments, including allergen-induced conditions or respiratory conditions and diseases.

### CROSS REFERENCE TO RELATED APPLICATIONS

The present application claims the benefit of Provisional Application Ser. No. 60/951,589, filed Jul. 24, 2007, the entire contents of which are hereby incorporated herein by reference.

### GOVERNMENT SUPPORT

This invention was supported in part with funding provided by NIH Grant No. HL65410 awarded by the National Institutes of Health. The government may have certain rights to this invention.

### FIELD OF INVENTION

The field of the present invention is immunotherapy, in particular, treatment of airway hyperresponsiveness (“AHR”) by targeting a subset of innate T cells.

### BACKGROUND OF THE INVENTION

Adaptive immunity depends on the synergistic actions of different lymphocyte-types. The best-studied example is the development of humoral responses to T-dependent antigens (Ag), which requires synergism of Ag-specific T and B cells. Likewise, the development of Ag-specific cytotoxic T lymphocytes is aided by Ag-specific T helper cells. In addition, the development of the Ag-specific immune responses appears to benefit from the synergistic action of innate T cells. However, it is not known whether innate T cells also synergize with one another during innate immune responses.

Airway hyperresponsiveness (“AHR”) is a characteristic feature of many airway diseases, for example asthma, and consists of an abnormality of the airways that allows them to narrow too easily and/or too much in response to a stimulus. In the pathogenesis of allergic airway diseases, antigen-specific memory T cells and allergen-specific antibodies are considered key. Studies in humans and rodents suggest important roles for classical CD4⁺ and CD8⁺αβ T cells in allergic inflammation. Recently, non-classical innate T cells including NKT cells and γδ T cells have also been implicated in allergic airway diseases.

NKT cells are innate αβ T cells with a restricted TCR repertoire, which co-express receptors of the NK-lineage, and participate in protective and pathological host responses and in allergic airway disease. In allergen-sensitized mice, allergen-nonspecific NKT cells expressing invariant TCRs (iNKT) increase airway inflammation and AHR, without a requirement for allergen-priming. iNKT cells express a semi-invariant TCRα chain (Vα14-Jα18) in association with Vβ8, Vβ7 and Vβ2, and recognize glycolipids presented by the MHC class I-like CD1d-molecule. They can be detected by staining with tetramerized CD1d/β₂M heterodimeric molecules complexed with the pharmacological ligand α-galactosyl-ceramide (αGalCer). iNKT cells in C57BL/6 mice also express the NK-receptor NK1.1, which is acquired during the final stages of their development.

Like iNKT cells, γδ T cells may also play a role in the lung pathology of allergen-sensitized mice, particularly in the development of AHR. In OVA-sensitized and challenged mice, γδ T cells expressing Vγ1 enhanced AHR, whereas cells expressing Vγ4 strongly suppressed AHR. The AHR regulatory γδ T cells had only minor effects on airway inflammation, however, and they did not appear to recognize OVA. Notably, young adult mice (6-12 wk) require γδ T cells for the development of AHR following sensitization and challenge with OVA, even though older mice (>6 months) develop AHR in the absence of γδ T cells.

The γδ T cells may depend in their functions on interactions with αβ T cells. While AHR-suppressive γδ T cells do not appear to require αβ T cells, it is not clear whether the AHR-enhancing γδ T cells depend on αβ T cells for this function.

Thus, there exists a need to gain a better understanding of the interactions between γδ T cells and αβ T cells (specifically, NKT cells) in the onset of AHR, so that effective methods for prevention and treatment of AHR can be developed.

### SUMMARY OF THE INVENTION

In one embodiment, the instant invention comprises a method to inhibit airway hyperresponsiveness (“AHR”) in an individual, comprising deleting, inactivating or inhibiting target innate γδ T cells comprising a murine Vγ1 chain or equivalent thereof in the individual species or a Vδ5 chain or equivalent thereof in the individual species, in an individual who has or is at risk of developing AHR.

In some embodiments, the instant invention comprises a method to inhibit AHR comprising deleting, inactivating or inhibiting target innate γδ T cells comprising a murine Vγ1/Vδ5 T cell receptor, or the equivalent thereof in the individual species. In some embodiments, the human equivalent is a T cell receptor comprising Vδ3. In some embodiments, the human equivalent is a T cell receptor comprising Vγ9/Vδ3.

In another embodiment, the instant invention comprises a method to inhibit AHR in a human, comprising deleting, inactivating or inhibiting target innate γδ T cells comprising a Vδ3 chain in a human who has or is at risk of developing AHR.

In some embodiments, the instant invention comprises a method to inhibit AHR in a human, comprising deleting, inactivating or inhibiting innate γδ T cells comprising a Vγ9/Vδ3 T cell receptor.

In some embodiments the AHR is associated with a condition selected from the group consisting of: asthma, chronic obstructive pulmonary disease, allergic bronchopulmonary aspergillosis, hypersensitivity pneumonia, eosinophilic pneumonia, emphysema, bronchitis, allergic bronchitis bronchiectasis, cystic fibrosis, tuberculosis, hypersensitivity pneumonitis, occupational asthma, sarcoid, reactive airway disease syndrome, interstitial lung disease, hyper-eosinophilic syndrome, rhinitis, sinusitis, exercise-induced asthma, pollution-induced asthma and parasitic lung disease. In some embodiments, the AHR is associated with a viral infection.

In some embodiments of the present invention, the target innate γδ T cells are deleted, inactivated or inhibited by selective leukophoresis.

In some embodiments, the target innate γδ T cells are deleted, inactivated or inhibited by administration of an agent that selectively targets the γδ T cells. In some embodiments the target innate γδ T cells are deleted, inactivated or inhibited by administration of an agent that selectively targets the Vγ chain expressed by the γδ T cells. In some embodiments the target innate γδ T cells are deleted, inactivated or inhibited by administration of an agent that selectively targets the Vδ chain expressed by the γδ T cells.

In some embodiments the agent is an antibody or antigen-binding fragment thereof. In some embodiments the antibody selectively targets the Vγ chain, the Vδ chain, or both the Vγ chain and the Vδ chain expressed by the γδ T cells. In some embodiments the agent is a soluble γδ T cell receptor identical or equivalent to that expressed by the γδ T cells to be deleted, inactivated or inhibited.

### BRIEF DESCRIPTION OF THE DRAWINGS

FIG. 1 illustrates that transfer of Vγ1⁺γδ T cells into γδ T cell-deficient mice can elicit AHR without allergen-priming.

FIG. 2 illustrates that both Vγ1⁺γδ T cells and endogenous NK1.1⁺ cells are required to elicit AHR.

FIG. 3 illustrates that iNKT cells synergize with Vγ1⁺γδ T cells to elicit AHR.

FIG. 4 illustrates that specifically Vγ1⁺Vδ5⁺γδ T cells are responsible for eliciting AHR.

### DETAILED DESCRIPTION

This invention generally relates to a method to inhibit airway hyperresponsiveness (AHR) by inhibiting a particular subset of γδ T cells, alone or in conjunction with the inhibition of NKT cells. More specifically, the present invention includes a method to inhibit AHR in a subject by inhibiting Vγ1/Vδ5 T cells or the subject's equivalent thereof (e.g., Vγ9/Vδ3 T cells in humans), preferably by administering to a subject an agent that inhibits the targeted γδ T cells. The method can additionally include the inhibition of NKT cells in the individual.

More particularly, it has been discovered that this subset of innate γδ T cells (i.e., does not require priming or allergen-selection), defined by a Vγ1/Vδ5 T cell receptor in mice, mediates airway hyperresponsiveness together with NKT cells in a well-defined mouse model for allergen-induced airway hyperresponsiveness that mimics the condition as it occurs in human allergic respiratory conditions. This is believed to be the first complete description of an innate AHR-regulating γδ T cell population with respect to Vγ usage of the TCR. The examples disclosed herein describe the use of mice sensitized and challenged with ovalbumin (OVA) to investigate the role of innate T cells in the development of allergic airway hyper-responsiveness (AHR). AHR, but not eosinophilic airway inflammation, was induced in T cell-deficient mice by small numbers of co-transferred γδ T cells and invariant NKT cells, whereas either cell-type alone was not effective. Only Vγ1⁺Vδ5⁺γδ T cells enhanced AHR. Surprisingly, OVA-specific αβ T cells were not required, revealing a pathway of AHR-development mediated entirely by innate T cells. These data indicate that lymphocytic synergism, which is key to the antigen-specific adaptive immune response, is also intrinsic to T cell-dependent innate responses. The present invention is particularly directed to the targeting of the specific innate γδ T cells identified herein, for the inhibition of AHR, particularly in humans. In some embodiments, NKT cells may be inhibited, but this is not a requirement of the present invention.

The equivalent γδ T cell subset in humans (e.g., Vγ9/Vδ3, or a γδ T cell receptor including a Vδ3 chain) are also innate enhancers of AHR and therefore, that inhibition of this specific subset of T cells in humans is a novel method to treat the condition of AHR in humans. To the best of the inventor's knowledge, the Vδ3 subset has not been previously implicated in human AHR or in human asthma. Since the present invention is directed to the inhibition of innate targets, rather than targets that are induced by priming or allergen-selection, the method is more predictable, easy to perform, and applicable for use in a wide variety of individuals with sensitivities to a wide variety of allergens. This wide applicability is largely due to the fact that innate targets are typically invariant (i.e., not dependent on genetic background of the individual or on hypersensitivity to particular allergens). Therefore, the method can be applied equally to individuals in need of treatment related to AHR.

Accordingly, one embodiment of the present invention relates to a method to inhibit or prevent AHR in an individual that has or is at risk of developing AHR by deleting, inactivating or inhibiting a specific target population of innate γδ T cells comprising a murine Vγ1 chain or equivalent thereof in the individual species or a Vδ5 chain or equivalent thereof in the individual species.

A “γδ T cell” is a distinct lineage of T lymphocytes found in mammalian species and birds that expresses a particular antigen receptor (i.e., T cell receptor or TCR) that includes a γ chain and a δ chain. γδ T cell receptors are composed of a heterodimer of a γ chain and a δ chain. Multiple different functional murine γ chains, murine δ chains, human γ chains, and human δ chains are known, and the sequences of the chains are publicly available (see, e.g., Arden, et al. Immunogenetics 1995; Allison and Garboczi, Molecular Immunol. 38: 1051-1061, 2002; Konihshofer and Chien, Current Opinion of Immunol. 18: 527-533, 2006). The γ and δ chains are distinguished from the α and β chains that make up the TCR of the perhaps more commonly referenced T cells known as “αβ T cells”. The γδ heterodimer of the γδ T cells is expressed on the surface of the T cell and, like the αβ heterodimer of αβ T cells, is associated with the CD3 complex on the cell surface. The γ and δ chains of the γδ T cell receptor should not be confused with the γ and δ chains of the CD3 complex. According to the present invention, the terms “T lymphocyte” and “T cell” can be used interchangeably herein.

Another embodiment of the present invention relates to a method to inhibit or prevent AHR in a human that has or is at risk of developing AHR by deleting, inactivating or inhibiting a specific target population of innate γδ T cells comprising a Vδ3 chain.

In preferred embodiments, in mice, the target population expresses a Vγ1/Vδ5 T cell receptor and in humans, the target population expresses a T cell receptor including a Vδ3 chain, and particularly, a Vγ9/Vδ3 T cell receptor. The method of the invention can be used to treat AHR in a variety of different diseases or conditions, including a variety of allergen-induced conditions, and particularly, in a variety of respiratory conditions or diseases. Also included in the method are compositions for use in any of the methods described herein, wherein the composition includes at least one agent useful for inhibiting the specific target population of innate γδ T cells in the individual, as described in detail herein.

NKT cells are innate αβ T cells with a restricted TCR repertoire, which co-express receptors of the NK-lineage, and participate in protective and pathological host responses and in allergic airway disease. In allergen-sensitized mice, allergen-nonspecific NKT cells expressing invariant TCRs (iNKT) increase airway inflammation and AHR, without a requirement for allergen-priming. iNKT cells express a semi-invariant TCRα chain (Vα14-Jα18) in association with Vβ8, Vβ7 and Vβ2, and recognize glycolipids presented by the MHC class I-like CD1d-molecule.

According to the invention, a variety of techniques can be used to delete, inactivate or inhibit the targeted innate γδ T cells. For example, in one aspect, a common ex vivo technique for removing components from the blood, and then returning the blood to the individual, known as leukophoresis, is used. The use of leukophoresis to selectively remove components from the blood, for example, by using binding agents such as antibodies or soluble receptors, has been described (e.g., see U.S. Pat. No. 6,379,708). In the present invention, blood can be removed from a patient to be treated, the blood treated to selectively remove the targeted γδ T cells, and then the blood is returned to the patient, free of γδ T cells that are contributing to disease processes. For example, γδ T cells, or selected subsets thereof, could be removed from the blood through the use of immobilized antibodies or other binding moieties that selectively bind to the targeted γδ T cell (e.g., antibodies that selectively bind to a particular Vδ chain, a particular Vγ chain, or both Vδ and Vγ chains), and thereby remove it from the blood. In some embodiments, γδ T cells, or selected subsets thereof, could be removed from the blood through the use of bispecific antibodies that are capable of selectively binding to a single targeted γδ T cell by binding to both Vδ and Vγ chains of the same cell. In some embodiments, the γδ T cells, or selected subsets thereof, could be removed from the blood through the use of bispecific antibodies that are capable of selectively binding to two different γδ T cells by binding to Vδ chain of one cell and Vγ chain of the other cell.

However, since the γδ T cells causing damage to the patient may be localized to a tissue or organ, other methods for the deletion, inactivation or inhibition of such cells may also be utilized and in some instances, may be preferable.

Another method of deleting, inactivating or inhibiting the targeted γδ T cells may include the administration of an agent that contacts the targeted γδ T cell, or a ligand with which the targeted γδ T cell interacts, resulting in the deletion of the γδ T cell, inactivation of the γδ T cell, or the inhibition of the γδ T cell. Inhibition can be achieved by directly inhibiting the activity of the targeted γδ T cell, or by inhibiting a biological activity of the γδ T cell by blocking or inhibiting the interaction of the T cell with, for example, its natural ligand. In the case of the innate γδ T cells of the invention, it will be most preferred to inhibit the activity by contacting the T cell with an agent that contacts the targeted γδ T cell, since this method takes advantage of the wide applicability of the targeting to most or all individuals.

According to the present invention, the phrase “selectively binds to” refers to the ability of an antibody, antigen-binding fragment or other binding partner of the present invention to preferentially bind to specified proteins. More specifically, the phrase “selectively binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen-binding fragment thereof in the absence of the antigen is considered to be background. Binding can be measured using a variety of methods standard in the art including enzyme immunoassays (e.g., ELISA), immunoblot assays, etc.). To “selectively target” a given moiety, such as a receptor, refers to the specific targeting of an agent, treatment or action to a specific moiety, such as by selective binding of an agent to the moiety, or by applying a protocol that will selectively act on the specified moiety and not substantially on another, different moiety.

Accordingly, in one aspect of the invention, the targeted γδ T cells are deleted, inactivated or inhibited by administration of an agent that selectively targets the γδ T cells having a specified Vγ and Vδ combination. In other embodiments, the agent can selectively target γδ T cells expressing murine TCR-Vγ1/Vδ5, or the equivalent thereof in the species of individual that is being treated. In humans, the equivalent target is believed to be human γδ T cells expressing Vδ3, and particularly, Vγ9/Vδ3. One type of agent that is particularly useful in these aspects of the invention includes, but is not limited to, antibodies or antigen-binding fragments thereof, or any binding protein or molecule (e.g., an aptamers) that selectively binds to the targeted TCR. It is noted that while the following discussion is made with respect to the targeted γδ T cells, the equivalent agents and techniques can be applied to the deletion, inactivation or inhibition of NKT cells, when such step is used in conjunction with the deletion, inactivation or inhibition of the target innate γδ T cells according to the invention.

Antibodies are characterized in that they comprise immunoglobulin domains and as such, they are members of the immunoglobulin superfamily of proteins. An antibody of the invention includes polyclonal and monoclonal antibodies, divalent and monovalent antibodies, bi- or multi-specific antibodies, serum containing such antibodies, antibodies that have been purified to varying degrees, and any functional equivalents of whole antibodies. Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)² fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention.

Genetically engineered antibodies of the invention include those produced by standard recombinant DNA techniques involving the manipulation and re-expression of DNA encoding antibody variable and/or constant regions. Particular examples include, chimeric antibodies, where the VH and/or VL domains of the antibody come from a different source to the remainder of the antibody, and CDR grafted antibodies (and antigen binding fragments thereof), in which at least one CDR sequence and optionally at least one variable region framework amino acid is (are) derived from one source and the remaining portions of the variable and the constant regions (as appropriate) are derived from a different source. Construction of chimeric and CDR-grafted antibodies are described, for example, in European Patent Applications: EP-A 0194276, EP-A 0239400, EP-A 0451216 and EP-A 0460617.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies may be produced according to the methodology of Kohler and Milstein (Nature 256:495-497, 1975). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

The invention also extends to non-antibody polypeptides, sometimes referred to as binding partners or antigen-binding polypeptides, that have been designed to bind specifically to a given protein. Examples of the design of such polypeptides, which possess a prescribed ligand specificity are given in Beste et al. (Proc. Natl. Acad. Sci. 96:1898-1903, 1999), incorporated herein by reference in its entirety.

Another useful agent in the methods of the invention is a soluble γδ T cell receptor or any competitive inhibitor that competitively inhibits the binding of the natural γδ T cell receptor to its ligand. According to the present invention, a competitive inhibitor of a γδ TCR is an inhibitor that binds to the same or similar epitope of a ligand (antigen) of the γδ TCR as the γδ TCR, such that binding of the γδ TCR to its ligand is inhibited. A competitive inhibitor may bind to the target ligand with a greater affinity for the target ligand than the γδ TCR.

Soluble γδ TCRs are described in detail in PCT Publication No. WO 03/060097, which is incorporated herein by reference in its entirety. According to the present invention, a “soluble” T cell receptor is a T cell receptor consisting of the chains of a full-length (e.g., membrane bound) receptor, except that, minimally, the transmembrane region of the receptor chains are deleted or mutated so that the receptor, when expressed by a cell, will not associate with the membrane. Most typically, a soluble receptor will consist of only the extracellular domains of the chains of the wild-type receptor (i.e., lacks the transmembrane and cytoplasmic domains). Various specific combinations of γ and δ chains are preferred for use in the soluble γδ T cell receptors, and particularly those corresponding to γδ T cell subsets that are known to exist in vivo, and more particularly those corresponding to γδ T cell subsets that are targeted for inhibition according to the invention. Preferably, soluble γδ T cell receptors comprise γ and δ chains derived from the same animal species (e.g., murine, human).

A soluble γδ T cell receptor useful in the invention typically is a heterodimer comprising a γ chain and a δ chain, but multimers (e.g., tetramers) comprising two different γδ heterodimers or two of the same γδ heterodimers are also contemplated for use in the present invention. As set forth above, preferably, γ and δ chains from the same species of mammal (e.g., murine, human) are combined to form a y6 heterodimer.

The invention also includes small molecule compounds that can serve as inhibitors of the target γδ T cell subset according to the invention (e.g., products of drug discovery). Such an agent can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the same building blocks) or by rational drug design. See for example, Maulik et al., 1997, Molecular Biotechnology: Therapeutic Applications and Strategies, Wiley-Liss, Inc., which is incorporated herein by reference in its entirety. Candidate compounds initially identified by drug design methods can be screened for the ability to modulate γδ T cell activity as described herein.

Any of the agents for the deletion, inactivation, or inhibition of γδ T cells (and/or NKT cells) described herein can be administered to an individual in the form of a therapeutic composition. Therapeutic compositions can also contain one or more pharmaceutically acceptable excipients, and/or one or more additional agents useful for treating a particular disease or condition (e.g., one or more agents suitable for use in treating an autoimmune disease, such as rheumatoid arthritis). As used herein, a pharmaceutically acceptable excipient refers to any substance suitable for delivering a therapeutic composition useful in the method of the present invention to a suitable in vivo or ex vivo site. Preferred pharmaceutically acceptable excipients are capable of maintaining a composition in a form that, upon arrival of the composition at a target cell, tissue, or site in the body, the therapeutic agent(s) is capable of acting in the intended manner. Suitable excipients of the present invention include excipients or formularies that transport, but do not specifically target a composition to a site (also referred to herein as non-targeting carriers). Examples of pharmaceutically acceptable excipients include, but are not limited to water, saline, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Suitable auxiliary substances include, for example, sodium acetate, sodium chloride, sodium lactate, potassium chloride, calcium chloride, and other substances used to produce phosphate buffer, Tris buffer, and bicarbonate buffer. Auxiliary substances can also include preservatives, such as thimerosal, m- or o-cresol, formalin and benzol alcohol.

The present invention includes the delivery of an agent or composition to an individual. The administration process can be performed ex vivo or in vivo. Ex vivo administration refers to performing part of the regulatory step outside of the patient, such as by removing cells from a patient, treating them to remove or inactivate the target γδ T cells, and then returning the remaining cells to the patient.

Administration of an agent or composition can be systemic, mucosal and/or proximal to the location of the target site (e.g., to the respiratory tract, including the lung, of an individual). The preferred routes of administration will be apparent to those of skill in the art, depending on the type of condition to be prevented or treated, the agent used, and/or the target cell population or tissue. Preferred methods of administration include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracranial, intraspinal, intraocular, aural, intranasal, oral, pulmonary administration, impregnation of a catheter, and direct injection into a tissue. Particularly preferred routes of administration include: intravenous, intraperitoneal, subcutaneous, intradermal, intranodal, intramuscular, transdermal, inhaled, intranasal, oral, intraocular, intraarticular, intracranial, and intraspinal. Parenteral delivery can include intradermal, intramuscular, intraperitoneal, intrapleural, intrapulmonary, intravenous, subcutaneous, atrial catheter and venal catheter routes. Aural delivery can include ear drops, intranasal delivery can include nose drops or intranasal injection, and intraocular delivery can include eye drops. Aerosol (inhalation) delivery can also be performed using methods standard in the art (see, for example, Stribling et al., Proc. Natl. Acad. Sci. USA 189:11277-11281, 1992, which is incorporated herein by reference in its entirety). For example, in one embodiment, a composition can be formulated into a composition suitable for nebulized delivery using a suitable inhalation device or nebulizer. Oral delivery can include solids and liquids that can be taken through the mouth.

According to the present invention, an effective administration protocol (i.e., administering an agent or therapeutic composition in an effective manner) comprises suitable dose parameters and modes of administration that result in the deletion, inactivation, or inhibition of the γδ T cells as described herein, preferably so that the individual receiving the treatment is provided with some benefit as a result of the administration. Preferably, the administration results in the alleviation or detectable improvement in the frequency of occurrence and/or severity of AHR, which provides a benefit to the individual who has a disease in which AHR is a symptom or condition. Effective dose parameters can be determined using methods standard in the art for a particular disease. Such methods include, for example, determination of survival rates, side effects (i.e., toxicity) and progression or regression of disease.

According to the present invention, “airway hyperresponsiveness” or “AHR” refers to an abnormality of the airways that allows them to narrow too easily and/or too much in response to a stimulus capable of inducing airflow limitation. AHR can be a functional alteration of the respiratory system caused by inflammation or airway remodeling (e.g., such as by collagen deposition). Airflow limitation refers to narrowing of airways that can be irreversible or reversible. Airflow limitation and/or airway hyperresponsiveness can be caused by collagen deposition, bronchospasm, airway smooth muscle hypertrophy, airway smooth muscle contraction, mucous secretion, cellular deposits, epithelial destruction, alteration to epithelial permeability, alterations to smooth muscle function or sensitivity, abnormalities of the lung parenchyma and/or infiltrative diseases in and around the airways. Many of these causative factors can be associated with inflammation. The present invention is directed to any airway hyperresponsiveness, including airway hyperresponsiveness that is associated with inflammation of the airways, eosinophilia and inflammatory cytokine production. Methods of measuring and monitoring AHR are discussed in detail below.

As used herein, to reduce airway hyperresponsiveness refers to any measurable reduction in airway hyperresponsiveness and/or any reduction of the occurrence or frequency with which airway hyperresponsiveness occurs in a patient. A reduction in AHR can be measured using any of the above-described techniques or any other suitable method known in the art. Preferably, airway hyperresponsiveness, or the potential therefore, is reduced, optimally, to an extent that the animal no longer suffers discomfort and/or altered function resulting from or associated with airway hyperresponsiveness. To prevent airway hyperresponsiveness refers to preventing or stopping the induction of airway hyperresponsiveness before biological characteristics of airway hyperresponsiveness as discussed above can be substantially detected or measured in a patient.

AHR can be measured by a stress test that comprises measuring an animal's respiratory system function in response to a provoking agent (i.e., stimulus). AHR can be measured as a change in respiratory function from baseline plotted against the dose of a provoking agent (a procedure for such measurement and a mammal model useful therefore are described in detail below in the Examples). Respiratory function can be measured by, for example, spirometry, plethysmography, peak flows, symptom scores, physical signs (i.e., respiratory rate), wheezing, exercise tolerance, use of rescue medication (i.e., bronchodialators) and blood gases. In humans, spirometry can be used to gauge the change in respiratory function in conjunction with a provoking agent, such as methacholine or histamine. In humans, spirometry is performed by asking a person to take a deep breath and blow, as long, as hard and as fast as possible into a gauge that measures airflow and volume. The volume of air expired in the first second is known as forced expiratory volume (FEV₁) and the total amount of air expired is known as the forced vital capacity (FVC). In humans, normal predicted FEV₁ and FVC are available and standardized according to weight, height, sex and race. An individual free of disease has an FEV₁ and a FVC of at least about 80% of normal predicted values for a particular person and a ratio of FEV₁/FVC of at least about 80%. Values are determined before (i.e, representing a mammal's resting state) and after (i.e., representing a mammal's higher lung resistance state) inhalation of the provoking agent. The position of the resulting curve indicates the sensitivity of the airways to the provoking agent.

The effect of increasing doses or concentrations of the provoking agent on lung function is determined by measuring the forced expired volume in 1 second (FEV₁) and FEV₁ over forced vital capacity (FEV₁/FVC ratio) of the mammal challenged with the provoking agent. In humans, the dose or concentration of a provoking agent (i.e., methacholine or histamine) that causes a 20% fall in FEV₁ (PD₂₀FEV₁) is indicative of the degree of AHR. FEV₁ and FVC values can be measured using methods known to those of skill in the art.

Pulmonary function measurements of airway resistance (R_{L}) and dynamic compliance (C_{dyn} or C_{L}) and hyperresponsiveness can be determined by measuring transpulmonary pressure as the pressure difference between the airway opening and the body plethysmograph. Volume is the calibrated pressure change in the body plethysmograph and flow is the digital differentiation of the volume signal. Resistance (R_{L}) and compliance (C_{L}) are obtained using methods known to those of skill in the art (e.g., such as by using a recursive least squares solution of the equation of motion). The measurement of lung resistance (R_{L}) and dynamic compliance (C_{L}) are described in detail in the Examples. It should be noted that measuring the airway resistance (R_{L}) value in a non-human mammal (e.g., a mouse) can be used to diagnose airflow obstruction similar to measuring the FEV₁ and/or FEV₁/FVC ratio in a human.

A variety of provoking agents are useful for measuring AHR values. Suitable provoking agents include direct and indirect stimuli. Preferred provoking agents include, for example, an allergen, methacholine, a histamine, a leukotriene, saline, hyperventilation, exercise, sulfur dioxide, adenosine, propranolol, cold air, an antigen, bradykinin, acetylcholine, a prostaglandin, ozone, environmental air pollutants and mixtures thereof. Preferably, Mch is used as a provoking agent. Preferred concentrations of Mch to use in a concentration-response curve are between about 0.001 and about 100 milligram per milliliter (mg/ml). More preferred concentrations of Mch to use in a concentration-response curve are between about 0.01 and about 50 mg/ml. Even more preferred concentrations of Mch to use in a concentration-response curve are between about 0.02 and about 25 mg/ml. When Mch is used as a provoking agent, the degree of AHR is defined by the provocative concentration of Mch needed to cause a 20% drop of the FEV₁ of a mammal (PC_{20methacholine}FEV₁). For example, in humans and using standard protocols in the art, a normal person typically has a PC_{20methacholine}FEV₁>8 mg/ml of Mch. Thus, in humans, AHR is defined as PC_{20methacholine}FEV₁<8 mg/ml of Mch.

According to the present invention, respiratory function can also be evaluated with a variety of static tests that comprise measuring an animal's respiratory system function in the absence of a provoking agent. Examples of static tests include, for example, spirometry, plethysmographically, peak flows, symptom scores, physical signs (i.e., respiratory rate), wheezing, exercise tolerance, use of rescue medication (i.e., bronchodialators) and blood gases. Evaluating pulmonary function in static tests can be performed by measuring, for example, Total Lung Capacity (TLC), Thoracic Gas Volume (TgV), Functional Residual Capacity (FRC), Residual Volume (RV) and Specific Conductance (SGL) for lung volumes, Diffusing Capacity of the Lung for Carbon Monoxide (DLCO), arterial blood gases, including pH, P_{O2} and P_{CO2} for gas exchange. Both FEV₁ and FEV₁/FVC can be used to measure airflow limitation. If spirometry is used in humans, the FEV₁ of an individual can be compared to the FEV₁ of predicted values. Predicted FEV₁ values are available for standard normograms based on the animal's age, sex, weight, height and race. A normal animal typically has an FEV₁ at least about 80% of the predicted FEV₁ for the animal. Airflow limitation results in a FEV₁ or FVC of less than 80% of predicted values. An alternative method to measure airflow limitation is based on the ratio of FEV₁ and FVC (FEV₁/FVC). Disease free individuals are defined as having a FEV₁/FVC ratio of at least about 80%. Airflow obstruction causes the ratio of FEV₁/FVC to fall to less than 80% of predicted values. Thus, an animal having airflow limitation is defined by an FEV₁/FVC less than about 80%.

An individual that has airway hyperresponsiveness is an individual in which airway hyperresponsiveness is measured or detected, such as by using one of the above methods for measuring airway hyperresponsiveness. To be associated with inflammation, the airway hyperresponsiveness is apparently or obviously, directly or indirectly associated with (e.g., caused by, a symptom of, indicative of, concurrent with) an inflammatory condition or disease (i.e., a condition or disease characterized by inflammation). Typically, such an inflammatory condition or disease is at least partially characterized by inflammation of pulmonary tissues. An animal that is at risk of developing airway hyperresponsiveness can be an animal that has a condition or disease which is likely to be associated with at least a potential for airway hyperresponsiveness, but does not yet display a measurable or detectable characteristic or symptom of airway hyperresponsiveness. The present invention is directed to controlling airway hyperresponsiveness by reducing or inhibiting the frequency and/or severity of acute incidents of airway hyperresponsiveness. An animal that is at risk of developing airway hyperresponsiveness also includes an animal that is identified as being predisposed to or susceptible to such a condition or disease.

Inflammation is typically characterized by the release of inflammatory mediators (e.g., cytokines or chemokines) which recruit cells involved in inflammation to a tissue. For example, a condition or disease associated with allergic inflammation is a condition or disease in which the elicitation of one type of immune response (e.g., a Th2-type immune response) against a sensitizing agent, such as an allergen, can result in the release of inflammatory mediators that recruit cells involved in inflammation in a mammal, the presence of which can lead to tissue damage and sometimes death. Airway hyperresponsiveness associated with allergic inflammation can occur in a patient that has, or is at risk of developing, any chronic obstructive disease of the airways, including, but not limited to, asthma, chronic obstructive pulmonary disease, allergic bronchopulmonary aspergillosis, hypersensitivity pneumonia, eosinophilic pneumonia, emphysema, bronchitis, allergic bronchitis bronchiectasis, cystic fibrosis, tuberculosis, hypersensitivity pneumonitis, occupational asthma, sarcoid, reactive airway disease syndrome, interstitial lung disease, hyper-eosinophilic syndrome, rhinitis, sinusitis, exercise-induced asthma, pollution-induced asthma and parasitic lung disease. Preferred conditions to treat using the method of the present invention include asthma, chronic obstructive disease of the airways, occupational asthma, exercise-induced asthma, pollution-induced asthma and reactive airway disease syndrome, with chronic obstructive disease of the airways and asthma being particularly preferred for treatment. Viral-induced inflammation typically involves the elicitation of another type of immune response (e.g., a Th1-type immune response) against viral antigens, resulting in production of inflammatory mediators the recruit cells involved in inflammation in a an animal, the presence of which can also lead to tissue damage. Airway hyperresponsiveness associated with viral-induced inflammation can occur in a patient that has, or is at risk of developing, an infection by a virus including, but not limited to, respiratory syncytial virus (RSV), parainfluenza virus (PIV), rhinovirus (RV) and adenovirus.

An “individual” is a vertebrate, preferably a mammal, more preferably a human. Mammals include, but are not limited to, farm animals, sport animals, pets, primates, mice and rats. The term “individual” can be used interchangeably with the term “animal”, “subject” or “patient”.

The following examples and experiments are provided for purposes of illustration and are not intended to limit the scope of the invention. Any variations which occur to the skilled artisan are intended to fall within the scope of the present invention. Each publication or other reference cited in the present application is incorporated herein by reference in its entirety.

### EXAMPLES

This example illustrates the synergistic action of γδ T cells and iNKT cells for eliciting AHR.

C57BL/6, B6.TCR-β^{-/-}, B6.TCR-β^{-/-}, and B6.TCR-β^{-/-/δ}^{-/ -} mice (The Jackson Laboratory, Bar Harbor, Me.) were maintained on ovalbumin free diet. The mice were 8-12 weeks old at the time of the experiments. All mice were cared for at National Jewish Medical and Research Center (Denver, Colo.), following guidelines for immune deficient animals. All experiments were conducted under a protocol approved by the Institutional Animal Care and Use Committee. Groups of mice were sensitized by i.p. injection of 20 μg ovalbumin grade V (Sigma-Aldrich, St. Louis, Mo.) emulsified in 2.25 mg aluminum hydroxide (Alumlmuject; Pierce Chemical, Rockford, Ill.) in a total volume of 100 ml on days 0 and 14 (2ip). Mice were challenged via the airways with OVA at a concentration of 10 mg/ml in saline for 20 min each on days 28, 29 and 30, by ultrasonic nebulization (particle size 1-5 mm; De Vilbiss, Somerset, Pa.). Lung resistance (RL) and dynamic compliance (Cdyn) were assessed 48 h after the last allergen challenge, and the mice were sacrificed to obtain tissues and cells for additional analysis. Immediately following measurements of AHR, lungs were lavaged, and Broncho-alveolar lavage (“BAL”) fluid was recovered. Total leukocyte numbers were measured (Coulter Counter; Coulter Electronics, Fla.). Differential cell counts were performed by light microscopy of cyto-centrifuged preparations (Cytospin2, Thermo Shandon, Waltham, Mass.), stained with Leukostat (Fisher Scientific, Pittsburgh, Pa.). For each sample, at least 200 cells were counted and differentiated by standard hematological procedures. All BAL cell counts and statistical analysis of differences are provided in Table 1 below. Anti-NK1.1 mAb PK136 was purified from hybridoma culture supernatant using a protein G-Sepharose affinity column (Pharmacia, Upsala, Sweden). Depletion of NK1.1+cells was achieved after injection of 200 μg of purified anti-NK1.1 mAb into the tail veins of mice, 3 days before the first OVA challenge and/or cell transfer. Depletion was monitored by immuno-cytofluorimetry. Donor spleens were homogenized, treated with Gey's solution for red blood cell-removal, and passed through nylon wool columns for T cell-enrichment. Non-adherent (“NAD”) cells were used for further purification. αβ T cells were purified from sensitized TCR-6^{-/-} mice. NAD cells were stained with FITC-conjugated anti-TCR-β mAb H57.597, with biotinylated anti NK1.1 mAb followed by PE-streptavidin or with PE-conjugated anti NK1.1 mAb, or with aGalCer-loaded CD1d/β₂M-tetramer conjugated to PE. Stained cells were sorted on a MoFlow cell sorter (Dako Cytomation, Inc., Denmark), and collected at a purity of >95%. Vγ1⁺γδ T cells were purified from the spleen of C57BL/6 or B6.TCR-β^{-/-} mice. NAD cells were stained with biotinylated anti Vγ1 mAb, and positively selected using streptavidin-conjugated magnetic beads (Streptavidin Microbeads, Miltenyi Biotec, Bergisch Gladbach, Germany). Repeated selection produced a cell population containing >90% viable Vγ1⁺ cells, as determined by two-color staining with anti TCR-δ GL3 and anti Vγ1 mAbs. The purified cells were washed and re-suspended in BSS, and injected via the tail vein into OVA-sensitized mice (B6.TCR-6-δ^{-/-} or B6.TCR-β^{-/-}/δ^{-/-}), less than 1 h prior to the first airway challenge. Airway responsiveness was assessed as a change in lung function after provocation with aerosolized methacholine (“MCh”). MCh aerosol was administered for 10 s (160 breaths/min, 0.5 ml tidal volume) in increasing concentrations. Maximum values of lung resistance (RL) and minimum values of dynamic compliance (Cdyn) were recorded and expressed as percent change from baseline after saline aerosol. Data are presented as means±standard error of the mean (SEM). The unpaired T test was used for two-group comparisons, and Two Way-ANOVA for analysis of differences in three or more groups. Pair-wise comparisons were performed using the post Bonferroni test. Statistically significant levels were set at a p value of <0.05.

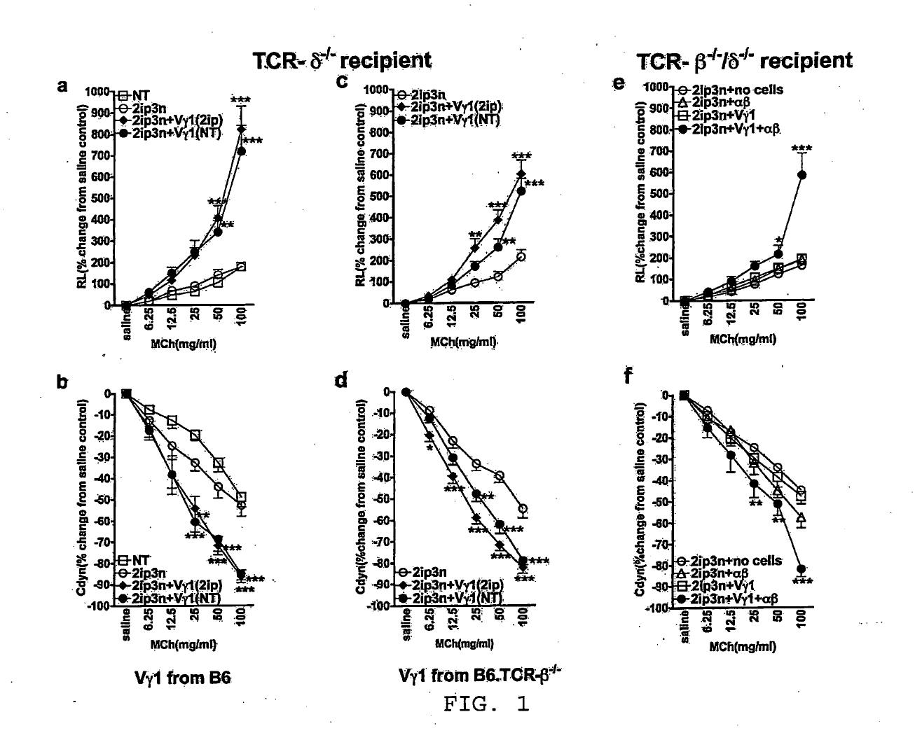

To test whether γδ T cells can enhance AHR without allergen-priming, 10⁴ purified Vγ1⁺γδ T cells were transferred from the spleen of untreated (NT) or sensitized (2ip) C57BL/6 donor mice into OVA sensitized γδ T cell-deficient recipients (B6.TCR-δ^{-/-}), just prior to OVA challenge. The results are summarized in FIG. 1a and b, which also show untreated recipients (NT) and recipients that were sensitized and challenged but did not receive cells (2ip3N). Results for each group are presented as means±SEM (n=12). Significant differences between 2ip3N and 2ip3N+Vγ1 groups are indicated: *P<0.05, **P<0.01, ***P<0.001. Without the transferred cells, the B6.TCR-δ^{-/-} mice showed only weak responses to inhaled MCh, based upon the changes seen in lung resistance (RL) and dynamic compliance (Cdyn). When reconstituted with Vγ1⁺ cells from either primed or unprimed donors, they developed AHR, indicating that the development of the AHR-enhancing γδ T cells does not require allergen-priming or help from allergen primed γδ T cells. In this and subsequent experiments, the AHR-enhancing γδ T cells had little or no effect on eosinophilic airway inflammation (Table 1).

Despite the absence of a priming-requirement, it remained possible that αβ T cells influence the normal development of these γδ T cells. Therefore, the cell-transfer experiment was repeated with Vγ1⁺γδ T cells derived from B6.TCR-β^{-/-} mice, in which they must develop in the absence of αβ T cells. (FIGS. 1c and d). Results for each group are presented as means±SEM (n=12). Significant differences between 2ip3N and 2ip3N+Vγ1 groups are indicated: *P<0.05, **P<0.01, ***P<0.001. These cells still enhanced AHR, indicating that αβ T cells are not required for the development of the AHR enhancing γδ T cells.

However, because the cell transfer recipients (B6.TCR-δ^{-/-} mice) contain αβ T cells, the possibility remained that αβ T cells are somehow involved in the AHR-enhancing effect of the transferred Vγ1⁺ cells. To examine this, OVA sensitized mice deficient in both αβ and γδ T cells (B6.TCR-β^{-/-}δ^{-/-}) were used as recipients (FIGS. 1e, f). OVA sensitized B6.TCR-β^{-/-}δ^{-/-} mice received 10⁴ splenic Vγ1⁺γδ T cells from sensitized B6.TCR-β^{-/-} donors and 2×10⁶ αβ T cells from sensitized B6.TCR-β^{-/-} donors (Vγ1+αβ), prior to airway challenge. Recipients that were sensitized and challenged but did not receive cells (2ip3N), or received only Vγ1⁺ cells (Vγ1) or only αβ T cells (αβ), are also shown. Results for each group are presented as means±SEM (n=4-9). Significant differences between sensitized and challenged mice, which received no cells, or Vγ1+ cells plus αβ T cells, are indicated: * P<0.05, **P<0.01, ***P<0.001. Transferred Vγ1+ cells alone failed to induce AHR, but produced a small AHR response when transferred together with αβ T cells, indicative of a role for αβ T cells in the effector-phase of the AHR-response. αβ T cells transferred alone had no effect. The comparatively weak AHR response in these mice despite a large number of transferred αβ T cells suggested that most αβ T cells were not effective.

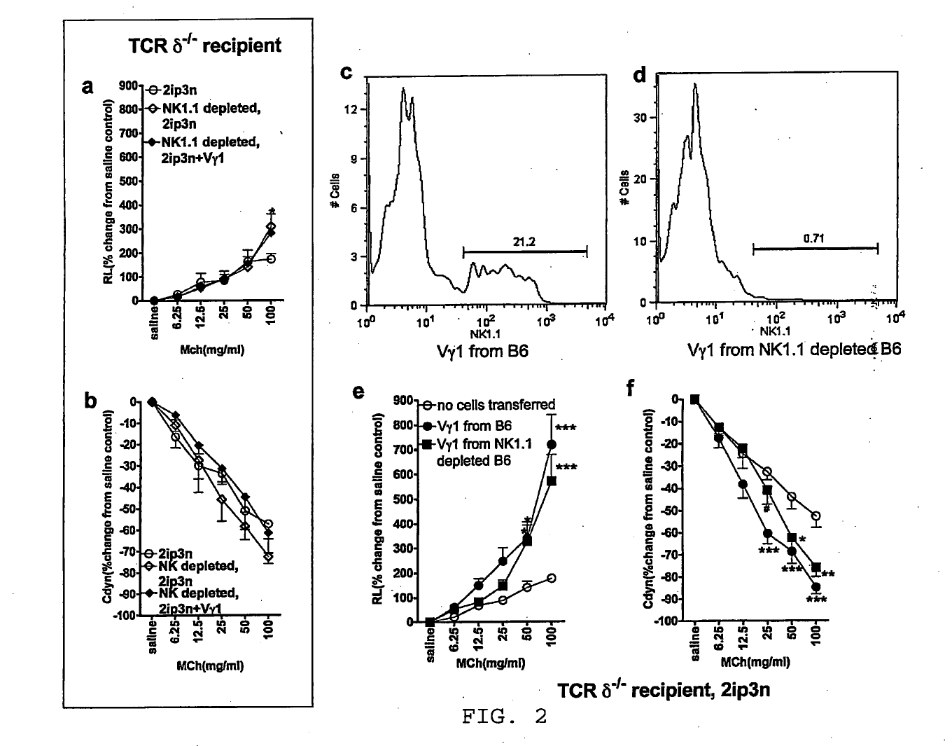

Because NKT cells reportedly mediate AHR, they might be the critical αβ T cell-component. To test this, the original experiment of transferring Vγ1⁺ cells into sensitized B6.TCR-δ^{-/-} mice was modified by treating the recipients first with NK1.1-depleting antibodies. FIG. 2 shows the results. AHR was monitored by measuring lung resistance and dynamic compliance. As seen in FIGS. 2a and b, Vγ1⁺ cells fail to restore AHR in γδ T cell-deficient mice pre-treated with anti NK1.1 mAb. OVA-sensitized B6.TCR-δ^{-/-} mice were treated with mAb PK136 (200 mg i.v.), received 10⁴ splenic Vγ1⁺γδ T cells 3 days later, and were then challenged via the airways (NK1.1-depleted, 2ip3N+Vγ1). Recipients that were only sensitized and challenged (2ip3N) and those that were sensitized and challenged and treated with the antibody (NK1.1-depleted, 2ip3N) are also shown. Results for each group are presented as means±SEM (n=4). Significant differences between 2ip3N and 2ip3N, NK1.1-depleted groups are indicated: * P<0.05.

FIGS. 2c and d show depletion of NK1.1⁺ cells within the Vγ1⁺ population upon treatment with antibody. C57BL/6 mice were treated with mAb PK136 (200 μg i.v.) and 3 days later, NAD splenocytes were stained for TCR-δ, Vγ1 and NK1.1. Cytofluorimetric analysis shows that approx. 20% of gated splenic Vγ1+γδ T cells express NK1.1 (FIG. 1c) and that the treatment with mAb PK136 removes most of these cells (FIG. 1d).

Because some γδ T cells themselves express NK1.1, it remained possible that residual antibody in the treated mice had inactivated the transferred γδ T cells. However, as seen in FIG. 2e and 2f, there is no significant difference in AHR-enhancement between total Vγ1⁺cells and NK1.1-depleted Vγ1⁺ cells. In this case, OVA-sensitized B6.TCR-δ^{-/-} mice received 10⁴ splenic Vγ1⁺γδ T cells from C57BL/6 donors prior to airway challenge. The cell donors were either untreated or received mAb PK136 i.v., 3 days prior to cell transfer (Vγ1 from B6 and Vγ1 from NK1.1-depleted B6). Recipients that were sensitized and challenged but did not receive cells, are also shown (no cells transferred). Results for each group are presented as means±SEM (n=7-8). Significant differences between mice that received no cells or Vγ1 cells are indicated: *P<0.05, **P<0.01 ***P<0.001. This result confirmed that both the transferred Vγ1⁺γδ T cells and endogenous NK1.1⁺ cells are required to elicit AHR in the B6.TCR-δ^{-/-} recipients.

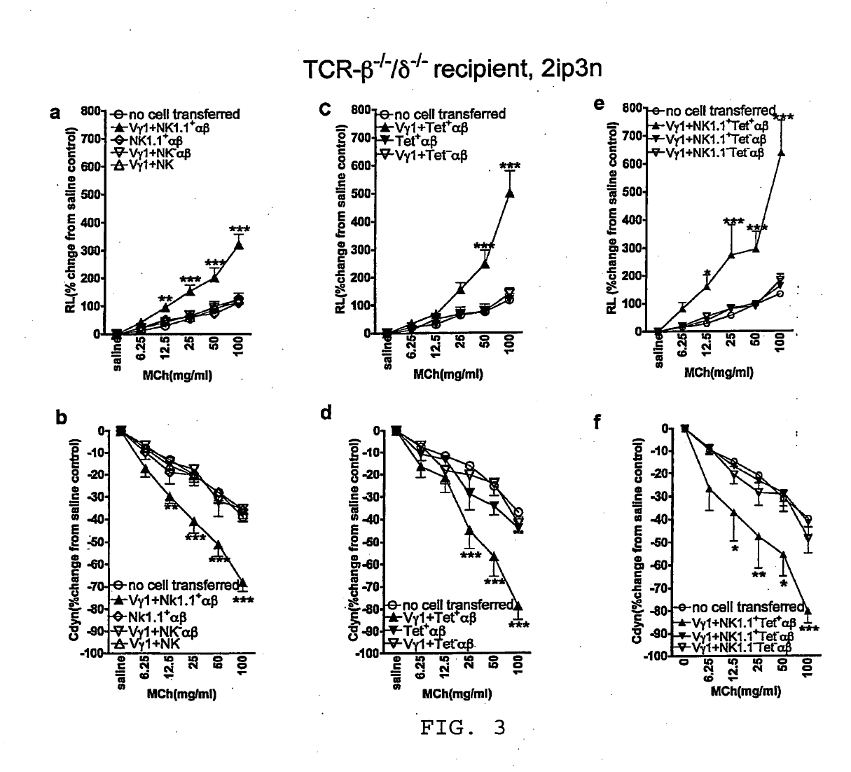

Although the requirement for both αβ T cells and NK1.1⁺ cells in γδ T cell-induced AHR might reflect a need for one type of cellular partner, this result could also indicate that more than one additional cell-type is involved, because NK1.1-expressing cells include non-T NK cells, as well as classical and non-classical NKT cells of the αβ T cell-lineage. To test the capacity of these different cell types, purified preparations of each type were co-transferred into OVA-sensitized B6.TCR-β^{-/-}δ^{-/-} mice. Results are summarized in FIG. 3.

FIGS. 3a and b show results obtained when OVA sensitized B6.TCR-β^{-/-}δ^{-/-} mice received 10⁴ splenic Vγ1⁺γδ T cells from sensitized B6.TCR-β^{-/-} donors and 2×10⁴ NK1.1⁺αβ T cells from sensitized B6.TCR-δ^{-/-} donors (Vγ¹⁺NK1.1⁺αβ), prior to airway challenge. Recipients that were sensitized and challenged but did not receive cells (no cell transferred), or which received 2×10⁴ NK1.1⁺αβ T cells (NK1.1⁺αβ) alone, Vγ1⁺ cells plus 2×10⁶ NK1.1⁻αβ T cells (Vγ1⁺NK1.1⁻αβ), or Vγ1+ cells plus 9×10⁴ NK1.1⁺ non-T cells (Vγ1⁺NK), are also shown. Results for each group are presented as means±SEM (n=6-7). Significant differences between mice that received no cells or Vγ1⁺ cells plus NK1.1⁺αβ T cells are indicated: **P<0.01, ***P<0.001. NK1.1⁺αβ T cells co-transferred with Vγ1⁺γδ T cells elicited a significant AHR-response, whereas neither NK1.1⁺αβ T cells alone, or either NK1.1⁻αβ T cells or NK1.1⁺ TCR-β⁻ NK cells co-transferred with the γδ T cells, enhanced AHR. This indicated that αβ NKT cells (invariant or other) synergize with Vγ1⁺γδ T cells to produce AHR.

To distinguish between the two, purified invariant NKT (“iNKT”) cells were transferred based on staining with αGalCer-loaded CD1d/β₂M tetramers together with Vγ1⁺γδ T cells. FIGS. 3c and d show results from experiments done with CD1d-tetramer⁺ αβ T cells (Vγ1⁺Tet⁺αβ). OVA sensitized B6.TCR-β^{-/-}δ^{-/-} mice received 10⁴ splenic Vγ1⁺γδ T cells from sensitized B6.TCR-β^{-/-} donors and 2×10⁴ CD1d-tetramer⁺ αβ T cells from sensitized B6.TCR-δ^{-/-} donors (Vγ¹⁺Tet⁺αβ), prior to airway challenge. Recipients that were sensitized and challenged but did not receive cells (no cell transferred), or which received 2×10⁴ Tet⁺αβT cells alone (Tet⁺αβ), or Vγ1⁺ cells plus 2×10⁴ Tet⁻αβT cells (Vγ1⁺Tet⁻αβ) are also shown. Results for each group are presented as means±SEM (n=4-5). Significant differences between mice that received no cells or Vγ1⁺ cells plus Tet⁺αβ T cells are indicated: ***P<0.001. The combination of Vγ1⁺γδ T cells with iNKT cells elicited a strong AHR response. However, iNKT cells alone, or tetramer negative αβ T cells together with the γδ T cells, had no effect.

Finally, to test whether iNKT cells are the only NK1.1⁺ cell population capable of synergy with the AHR enhancing γδ T cells, we compared the effect of NK1.1⁺αβ T cells that were either tetramer-positive or -negative. FIGS. 3e and f show results from these experiments. OVA sensitized B6.TCR-β^{-/-}δ^{-/-} mice received 10⁴ splenic Vγ1⁺γδ T cells from sensitized B6.TCR-β^{-/-} donors and 2×10⁴ NK1.1⁺ CD1d-tetramer⁺ αβ T cells from sensitized B6.TCR-δ^{-/-} donors (Vγ¹⁺NK1.1⁺Tet⁺αβ), prior to airway challenge. Recipients that were sensitized and challenged but did not receive cells (no cell transferred), or which received Vγ1⁺ cells plus 2×10⁴ NK1.1⁺Tet⁻αβ T cells (Vγ1⁺ NK1.1⁺Tet αβ), or Vγ1⁺ cells plus 2×10⁴ NK1.1⁻Tet⁻αβT cells (Vγ1⁺NK1.1⁻Tet⁻αβ) are also shown. Results for each group are presented as means±SEM (n=5-6). Significant differences between mice that received no cells or Vγ1⁺ cells plus NK1.1⁺Tet⁺αβ T cells are indicated: *P<0.05, **P<0.01, ***P<0.001. Only NK1.1⁺αβ T cells that were tetramer positive induced AHR when co-transferred with Vγ1+γδ T cells, suggesting that the synergism only involves iNKT cells.

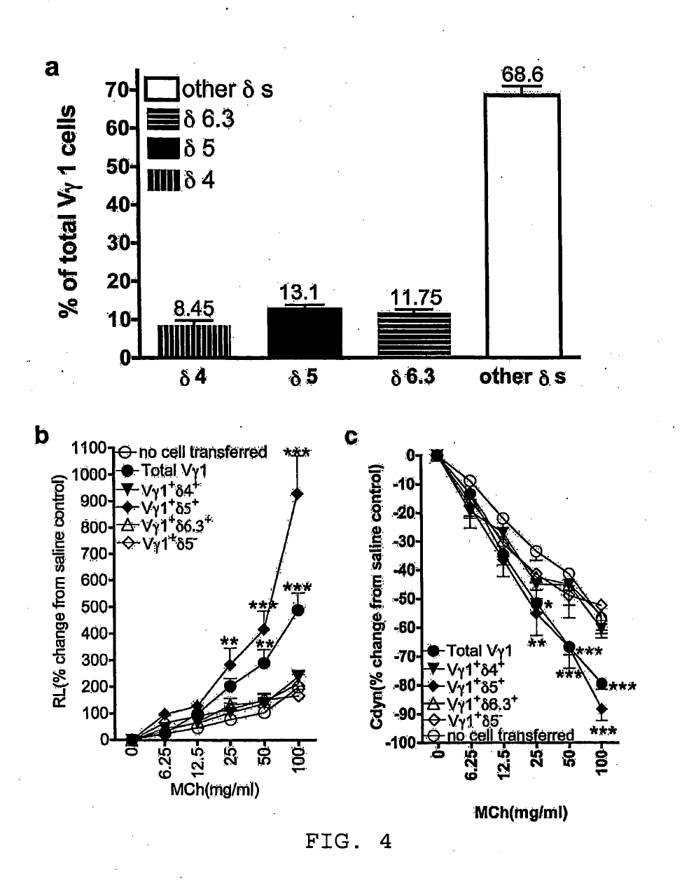

iNKT cells have a limited TCR repertoire. However, Vγ1⁺ cells can express several Vδ genes. FIG. 4a shows Vδ-expression among Vγ1⁺γδ T cells in B6.TCR-β^{-/-} spleen. NAD splenocytes of adult B6.TCR-β^{-/-} mice were stained with antibodies against Vγ1, TCR-δ and several Vδs, and analyzed cyto-fluorimetrically. Results are presented as means±SEM (n=5-11). Next, Vγ1⁺ cells expressing individual Vδs were purified and their ability to induce AHR was examined. OVA-sensitized B6.TCR-δ^{-/-} mice received 10⁴ sorted Vγ1⁺γδ T cells from B6.TCR-β^{-/-} spleen, expressing the indicated V6s, prior to airway challenge (2ip3N+Vγ1). FIGS. 4b and c illustrate the results. Recipients that were sensitized and challenged but did not receive cells (no cell transferred), are also shown. Results for each group are presented as means±SEM (n=5-8). Significant differences between mice that received no cells or Vγ1⁺ cells are indicated: **P<0.01, ***P<0.001. Only Vδ1⁺Vδ5⁺ cells induced AHR, indicating that the TCR repertoire of AHR-enhancing innate γδ T cells is highly limited as well. Thus, as far as T cells are concerned, the combined action of two innate T cell-types appears to be sufficient to mediate AHR in the OVA-model.

With regard to AHR in the OVA models, B cells were found to be required in at least one model, as well as in a hapten-model of non-atopic asthma. However, neither αβ T cells nor γδ T cells are always required for the development of AHR, and the role of NKT cells has been questioned. This example confirms that innate lymphocyte populations, including iNKT cells, can play a role in the allergic airway disease induced by OVA. In this role, the innate lymphocytes appear to be independent of the allergen-specific T cells but they cannot fully replace them. We had previously reported that, although γδ T cells can have a strong effect on AHR, they do not substantially alter airway cytokines or eosinophilic inflammation. This difference was maintained in the current study, because γδ T cells whether or not they were co-transferred with iNKT cells did not induce substantial changes in airway eosinophils compared with controls that did not receive transferred cells (Table 1). However, treatment with the anti NK1.1 mAb reduced airway eosinophilic infiltrations.

These findings show that in the absence of allergen-specific T cells, neither γδ T cells nor iNKT cells alone can mediate AHR. iNKT cells have been previously implicated in allergic airway inflammation and AHR even in mice lacking CD4⁺ allergen-specific αβ T cells, but a dependence on γδ T cells was not noted. In contrast to these studies, and other studies with conventional T cells, we have transferred much smaller numbers of cells (10⁴ Vγ1⁺γδ T cells and 2×10⁴ iNKT cells), which may be crucial in detecting the mutual dependence of these cell-types. If quantities of cytokines (e.g. IL-13) are critical in the development of eosinophilic inflammation, the small numbers of transferred innate T cells might also explain why no effect of these cells on eosinophilic airway infiltration was seen (Table 1).

Cooperation between different lymphocyte-types has long been recognized as a hallmark of the adaptive, antigen-specific immune response, exemplified by the classical mechanism of direct T-B cooperation, but also including synergistic interactions between antigen-specific T cells and innate lymphocyte-types. Whether such interactions are direct or involve cellular intermediates such as the multifunctional dendritic cells, all appear to benefit from the different functional potentials of the participating lymphocytes. The example of a synergism between γδ T cells and iNKT cells described here probably is based as well upon a complementary functional potential of these innate lymphocytes. The observation that Vγ1⁺Vδ6⁺γδ T cells are not AHR-enhancing is consistent with this notion. This subset of the Vγ1⁺ population contains γδ T cells with NKT-like properties and thus would not be expected to complement the iNKT cells.

This example may represent a specific case of the synergism between γδ and αβ T cells. In addition, because not only iNKT but also the AHR-enhancing γδ T cells could be derived from unprimed donors and express a very limited TCR-repertoire in this example, the results suggest that lymphocytic synergism might be a mechanism employed not only by adaptive but also by innate T-dependent immune responses.

TABLE 1
Cell numbers (×1000) and percentages, in the Bal fluid of cell transfer-recipients (mean ± SEM)

| | Group | Total cell | Eosinophil | Macrophage |
|---|---|---|---|---|
| Figure | (treatment) | number | (% of total) | (% of total) |
| FIG. 1 | NT | 392 ± 50 | 0 ± 0 (0 ± 0) | 387 ± 60 (100 ± 0) |
| (a, b) | 2ip3n | 212 ± 36** | 106 ± 31 (47 ± 10)* | 100 ± 25** (40 ± 10)** |
| | 2ip3n + Vγ1(2ip) | 177 ± 11** | 103 ± 10 (59 ± 4)* | 63 ± 8** (36 ± 5)** |
| | 2ip3n + Vγ1(NT) | 205 ± 108** | 36 ± 9 (29 ± 7)^{#} | 167 ± 105** (70 ± 7)**^{##} |
| FIG. 1 | 2ip3n | 192 ± 30 | 95 ± 22 (44 ± 10) | 92 ± 9 (53 ± 11) |
| (c, d) | 2ip3n + Vγ1(2ip) | 332 ± 101 | 172 ± 6 (44 ± 7) | 117 ± 29 (47 ± 9) |
| | 2ip3n + Vγ1(NT) | 150 ± 15^{##} | 82 ± 17 (52 ± 8) | 58 ± 10 (42 ± 9)^{#} |
| FIG. 1 | 2ip3n + no cells | 142 ± 24 | 0 ± 0 | 141 ± 24 |
| (e.f) | 2ip3n + αβ | 182 ± 54 | 4 ± 4 | 177 ± 50 |
| | 2ip3n + Vγ1 | 240 ± 92 | 1 ± 1 | 237 ± 92 |
| | 2ip3n + Vγ1 + αβ | 141 ± 19 | 0 ± 0 | 139 ± 18 |
| FIG. 2 | 2ip3n | 280 ± 51 | 140 ± 56 (48 ± 41) | 131 ± 45 (50 ± 14) |
| (a, b) | NK1.1 depleted | 276 ± 35 | 37 ± 28 (12 ± 9)* | 240 ± 31* (88 ± 8)** |
| | 2ip3n | | | |
| | NK1.1 depleted, | 215 ± 42 | 27 ± 10 (14 ± 7)* | 187 ± 42* (86 ± 7)** |
| | 2ip3n + VγI | | | |
| FIG. 2 | 2ip3n | 212 ± 36 | 106 ± 31 (47 ± 10) | 100 ± 25 (40 ± 10) |
| (e, f) | Vγ1 from B6 | 205 ± 108 | 36 ± 9 (29 ± 7) | 167 ± 105 (70 ± 7) |
| | Vγ1 from NK1.1 | 164 ± 22 | 20 ± 11 (12 ± 6)*** | 144 ± 25 (88 ± 6)*** |
| | Depleted B6 | | | |
| FIG. 3 | No cell | 142 ± 24 | 0 ± 0 | 141 ± 24 |
| (a, b) | transferred | | | |
| | Vγ1 + NK1.1⁺ αβ | 136 ± 13 | 0 ± 0 | 136 ± 13 |
| | NK1.1⁺αβ | 109 ± 18 | 2 ± 1 | 106 ± 18 |
| | Vγ1 + NK1.1⁻αβ | 154 ± 20 | 0 ± 0 | 154 ± 20 |
| | Vγ1 + NK | 148 ± 15 | 0 ± 0 | 148 ± 15 |
| FIG. 3 | No cell | 142 ± 24 | 0 ± 0 | 141 ± 24 |
| (c, d) | transferred | | | |
| | Vγ1 + Tet⁺αβ | 155 ± 26 | 0 ± 0 | 155 ± 26 |
| | Tet⁺αβ | 97 ± 15 | 0 ± 0 | 97 ± 15 |
| | VγI + Tet⁻αβ | 142 ± 37 | 0 ± 0 | 142 ± 37 |
| FIG. 3 | No cell | 142 ± 24 | 0 ± 0 | 141 ± 24 |
| (e, f) | transferred | | | |
| | Vγ1 + | 96 | 0 | 96 |
| | NK1.1⁺Tet⁺αβ | | | |
| | Vγ1 + | 290 ± 12 | 0 ± 0 | 290 ± 120 |
| | NK1.1⁺Tet⁻αβ | | | |
| | Vγ1 + | 115 ± 17 | 2 ± 2 | 113 ± 17 |
| | NK1.1⁻Tet⁻αβ | | | |
| FIG. 4 | No cell | 212 ± 36 | 106 ± 31 (47 ± 10) | 100 ± 25 (40 ± 10) |
| (b, c) | transferred | | | |
| | Total Vγ1 | 150 ± 15 | 82 ± 17 (52 ± 8) | 58 ± 10 (42 ± 9) |
| | Vγ1⁺δ4⁺ | 200 | 65 | 141 |
| | Vγ1⁺δ5⁺ | 194 ± 36 | 100 ± 21 (39 ± 7) | 159 ± 29 (60 ± 7) |
| | Vγ1⁺δ6.3⁺ | 106 ± 23 | 16 ± 5* (14 ± 3) | 87 ± 18 (84 ± 4) |
| | Vγ1⁺δ5⁻ | 125 ± 14 | 37 ± 19 (26 ± 12) | 88 ± 15 (73 ± 12) |

| | Lymphocyte | Neutrophil | | |
|---|---|---|---|---|
| | (% of | (% of | Number | Statistical |
| Figure | total) | total) | of mice | significant |
| FIG. 1 | 6 ± 3 (0 ± 0) | 1 ± 1 (0 ± 0) | 5 | *p < 0.05, |
| (a, b) | | | | **p < 0.01 vs NT |
| | | | | group |
| | 4 ± 2 (2 ± 1) | 3 ± 1 (1 ± 0) | 8 | |
| | 2 ± 1 (1 ± 0) | 8 ± 3 (4 ± 1) | 8 | *P < 0.05, |
| | | | | **P < 0.01 vs |
| | | | | Vγ1 (2ip) group |
| | 1 ± 0 (1 ± 0) | 2 ± 1 (1 ± 1) | 7 | |
| FIG. 1 | 3 ± 1 (1 ± 0) | 3 ± 1 (1 ± 0) | 11 | *P < 0.05, |
| (c, d) | | | | **P < 0.01 vs |
| | | | | Vγ1 (2ip) group |
| | 18 ± 7 (4 ± 1) | 24 ± 19 (4 ± 2) | 8 | |
| | 5 ± 1 (3 ± 1) | 5 ± 2 (3 ± 1) | 8 | |
| FIG. 1 | 0 ± 0 | 1 ± 1 | 5 | |
| (e.f) | 0 ± 0 | 0 ± 0 | 4 | |
| | 0 ± 0 | 2 ± 1 | 6 | |
| | 1 ± 0 | 1 ± 1 | 6 | |
| FIG. 2 | 6 ± 4 (2 ± 1) | 4 ± 2 (1 ± 1) | 4 | *p < 0.05, |
| (a, b) | | | | **P < 0.01 vs 2ip3n |
| | | | | group |
| | 0 ± 0 (0 ± 0) | 0 ± 0 (0 ± 0) | 3 | |
| | 0 ± 0 (0 ± 0) | 0 ± 0 (0 ± 0) | 4 | |
| FIG. 2 | 4 ± 2 (2 ± 1) | 3 ± 1 (1 ± 0) | 8 | ***P < 0.01 vs |
| (e, f) | | | | 2ip3n group |
| | 1 ± 0 (1 ± 0) | 2 ± 1 (1 ± 1) | 7 | |
| | 0 ± 0 (0 ± 0) | 0 ± 0 (0 ± 0) | 6 | |
| FIG. 3 | 0 ± 0 | 1 ± 1 | 5 | |
| (a, b) | | | | |
| | 0 ± 0 | 0 ± 0 | 7 | |
| | 0 ± 0 | 0 ± 0 | 4 | |
| | 0 ± 0 | 0 ± 0 | 3 | |
| | 0 ± 0 | 0 ± 0 | 5 | |
| FIG. 3 | 0 ± 0 | 1 ± 1 | 5 | |
| (c, d) | | | | |
| | 0 ± 0 | 0 ± 0 | 5 | |
| | 0 ± 0 | 0 ± 0 | 4 | |
| | 0 ± 0 | 0 ± 0 | 5 | |
| FIG. 3 | 0 ± 0 | 1 ± 1 | 5 | |
| (e, f) | | | | |
| | 0 | 0 | 1^{&} | |
| | 0 ± 0 | 0 ± 0 | 4 | |
| | 0 ± 0 | 0 ± 0 | 4 | |
| FIG. 4 | 4 ± 2 (2 ± 1) | 3 ± 1 (1 ± 0) | 8 | *P < 0.01 vs no cell |
| (b, c) | | | | transferred group |
| | 5 ± 1 (3 ± 1) | 5 ± 2 (3 ± 1) | 8 | |
| | 2 | 5 | 2^{&} | |
| | 1 ± 1 (0 ± 0) | 4 ± 2 (1 ± 1) | 4 | |
| | 1 ± 1 (0 ± 0) | 2 ± 1 (1 ± 1) | 4 | |
| | 0 ± 0 (0 ± 0) | 0 ± 0 (0 ± 0) | 5 | |

^{&}In these cases, not all of the animals that were analyzed for lung function remained available for BAL analysis.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

### REFERENCES

1. Claman, H. N., E. A. Chaperon, and R. F. Triplett. 1966. Immunocompetence of transferred thymus-marrow cell combinations. J. Immunol. 97:828-832.
2. Castellino, F., A. Y. Huang, G. Altan-Bonnet, S. Stoll, C. Scheinecker, and R. N. Germain. 2006. Chemokines enhance immunity by guiding naive CD8+T cells to sites of CD4+T cell-dendritic cell interaction. Nature 440:890-895.
3. Munz, C., R. M. Steinman, and S. Fujii. 2005. Dendritic cell maturation by innate lymphocytes: coordinated stimulation of innate and adaptive immunity. J. Exp. Med. 202:203-207.
4. Wills-Karp, M. 1999. Immunologic basis of antigen-induced airway hyperresponsiveness. Annu. Rev. Immunol. 17:255-281.
5. Robinson, D. S., O. Hamid, S. Ying, A. Tsicopoulos, J. Barkans, A. M. Bentley, C. Corrigan, S. R. Durham, and A. B. Kay. 1992. Predominant TH2-like bronchoalveolar T-lymphocyte population in atopic asthma. N. Engl. J. Med. 326:298-304.
6. Hamelmann, E., A. Oshiba, J. Paluh, K. Bradley, J. Loader, T. A. Potter, G. L. Larsen, and E. W. Gelfand. 1996. Requirement for CD8+T cells in the development of airway hyperresponsiveness in a murine model of airway sensitization. J Exp Medicine 183:1 719-1 729.
7. Akbari, O., P. Stock, E. Meyer, M. Kronenberg, S. Sidobre, T. Nakayama, M. Taniguchi, M. J. Grusby, R. H. DeKruyff, and D. T. Umetsu. 2003. Essential role of NKT cells producing IL-4 and IL-13 in the development of allergen-induced airway hyperreactivity. Nature Medicine 9:582-588.
8. Lisbonne, M., S. Diem, A. de Castro Keller, J. Lefort, L. M. Araujo, P. Hachem, J. M. Fourneau, S. Sidobre, M. Kronenberg, M. Taniguchi, P. Van Endert, M. Dy, P. Askenase, M. Russo, B. B. Vargaftig, A. Herbelin, and M. C. Leite-de-Moraes. 2003. Cutting edge: invariant V alpha 14 NKT cells are required for allergen induced airway inflammation and hyperreactivity in an experimental asthma model. J. Immunol. 171:1637-1641.
9. Zuany-Amorim, C., C. Ruffle, S. Haile, B. B. Vargaftig, P. Pereira, and M. Pretolani. 1998. Requirement for γδ T cells in allergic airway inflammation. Science 280:1265-1267.
10. Lahn, M., A. Kanehiro, K. Takeda, A. Joetham, J. Schwarze, G. Koehler, R. O'Brien, E. W. Gelfand, and W. Born. 1999. Negative regulation of airway responsiveness that is dependent on γδ T cells and independent of αβ T cells. Nature Medicine 5:1150-1156.
11. Das, Y., P. Eynott, R. Jupp, A. Bothwell, L. Van Kaer, Y. Shi, and G. Das. 2006. Natural killer T cells and CD8+T cells are dispensable for T cell-dependent allergic airway inflammation (Correspondence). Nature Medicine 12:1345-1347.
12. Bendelac, A., P. B. Savage, and L. Teyton. 2007. The biology of NKT cells. Annu. Rev. Immunol. 25:297-336.
13. Kinjo, Y., and M. Kronenberg. 2005. Valphal4i NKT cells are innate lymphocytes that participate in the immune response to diverse microbes. J. Clin. Immunol. 25:522-533.
Yu, K. O., and S. A. Porcelli. 2005. The diverse functions of CD1d-restricted NKT cells and their potential for immunotherapy. Immunol. Lett. 100:42-55.
15. Akbari, O., J. L. Faul, E. G. Hoyte, G. J. Berry, J. Wahlstrom, M. Kronenberg, R. H. DeKruyff, and D. T. Umetsu. 2006. CD4+ invariant T-cell-receptor+natural killer T cells in bronchial asthma. N. Engl. J. Med. 354:1117-1129.
16. Bendelac, A., M. N. Rivera, S.-H. Park, and J. H. Roark. 1997. Mouse CD1-specific NK1 T cells: development, specificity, and function. Annu. Rev. Immunol. 15:535-562.
17. Matsuda, J. L., O. V. Naidenko, L. Gapin, T. Nakayama, M. Taniguchi, C. R. Wang, Y. Koezuka, and M. Kronenberg. 2000. Tracking the response of natural killer T cells to a glycolipid antigen using CDld tetramers. J. Exp. Med. 192:741-754.
18. Matsuda, J. L., and L. Gapin. 2005. Developmental program of mouse V_{—}14i NKT cells. Current Opinion in Immunology 17:122-130.
19. Hahn, Y.-S., C. Taube, N. Jin, L. Sharp, J. M. Wands, M. Kemal Aydintug, M. Lahn, S. A. Huber, R. L. O'Brien, E. W. Gelfand, and W. K. Born. 2004. Different potentials of γδ T cell subsets in regulating airway responsiveness: Vγ1+ cells, but not Vγ4+ cells, promote airway hyperreactivity, TH2 cytokines, and airway inflammation. J. Immunol. 172:2894-2902.
20. Lahn, M., A. Kanehiro, K. Takeda, J. Terry, Y.-S. Hahn, M. K. Aydintug, A. Konowal, K. Ikuta, R. L. O'Brien, E. W. Gelfand, and W. K. Born. 2002. MHC class I-dependent Vγ4+ pulmonary T cells regulate αβ T cell-independent airway responsiveness. Proc. Natl. Acad. Sci (USA) 99:8850-8855.
21. Hahn, Y.-S., C. Taube, N. Jin, K. Takeda, J.-W. Park, J. M. Wands, M. K. Aydintug, C. L. Roark, M. Lahn, R. L. O'Brien, E. W. Gelfand, and W. K. Born. 2003. Vγ4+T cells regulate airway hyperreactivity to methacholine in ovalbuminsensitized and challenged mice. J. Immunol. 171:3170-3178.
22. Jin, N., C. Taube, L. Sharp, Y.-S. Hahn, X. Yin, J. M. Wands, C. L. Roark, R. L. O'Brien, E. W. Gelfand, and W. K. Born. 2005. Mismatched antigen prepares gd T cells for suppression of airway hyperresponsiveness. J. Immunol. 174:2671-2679.
23. Kasahara, Y., C. H. Chen, and M. D. Cooper. 1993. Growth requirements for avian γδ T cells include exogenous cytokines, receptor ligation and in vivo priming. Eur. J. Immunol. 23:2230-2236.
24. Pereira, P., D. Gerber, S. Y. Huang, and S. Tonegawa. 1995. Ontogenic development and tissue distribution of Vγ1-expressing γ/δT lymphocytes in normal mice. J. Exp. Med. 182:1921-1930.
25. Heilig, J. S., and S. Tonegawa. 1986. Diversity of murine gamma genes and expression in fetal and adult T lymphocytes. Nature 322:836-840.
26. Silva-Santos, B., D. J. Pennington, and A. C. Hayday. 2005. Lymphotoxinmediated regulation of γδ cell differentiation by αβ T cell progenitors. Science 307:925-928.
27. Sentman, C. L., J. Hackett Jr., T. A. Morre, M. M. Tutt, M. Bennett, and V. Kumar. 1989. Pan natural killer cell monoclonal antibodies and their relationship to the NK1.1 antigen. Hybridoma 8:605-614.
28. Vicari, A. P., S. Mocci, P. Openshaw, A. O'Garra, and A. Zlotnik. 1996. Mouse γδ TCR⁺NK1.1⁺ thymocytes specifically produce interleukin-4, are major histocompatibility complex class I independent, and are developmentally related to ab TCR+NK1.1+ thymocytes. Eur. J. Immunol. 26:1424-1429.
29. Azuara, V., J. P. Levraud, M. P. Lembezat, and P. Pereira. 1997. A novel subset of adult gamma delta thymocytes that secretes a distinct pattern of cytokines and expresses a very restricted T cell receptor repertoire. Eur. J. inmunol. 27:544-553.
30. Godfrey, D. I., H. R. MacDonald, M. Kronenberg, M. J. Smyth, and L. Van Kaer. 2004. NKT cells: what's in a name? Nat. Rev. inmunol. 4:231-237.
31. Hamelmann, E., A. T. Vella, A. Oshiba, J. W. Kappler, P. Marrack, and E. W. Gelfand. 1997. Allergic airway sensitization induces T cell activation but not airway hyperresponsiveness in B cell-deficient mice. Proc. Natl. Acad. Sci., USA 94:1350-1355.
32. Kawikova, I., V. Palival, M. Szczepanik, A. Itakura, M. Fukui, R. A. Campos, G. P. Geba, R. J. Homer, B. P. Iliopoulou, J. S. Pober, R. F. Tsuji, and P. W. Askenase. 2004. Airway hyper-reactivity mediated by B-1 cell immunoglobulin M antibody generating complement C5a at 1 day post-immunization in a murine hapten model of non-atopic asthma. Immunology 113:234-245.
33. Vijayanand, P., G. Seumois, C. Pickard, R. M. Powell, G. Angco, D. Sammut, S. D. Gadola, P. S. Friedman, and R. Djukanovic. 2007. Invariant natural killer T cells and chronic obstructive pulmonary disease. N. Engl. J. Med. 356:1410-1422.
34. Miyahara, N., B. J. Swanson, K. Takeda, C. Taube, S. Miyahara, T. Kodama, A. Dakhama, V. L. Ott, and E. W. Gelfand. 2004. Effector CD8+T cells mediate inflammation and airway hyper-responsiveness. Nature Medicine 10:865-869.
35. McMenamin, C., C. Pimm, M. McKersey, and P. G. Holt. 1994. Regulation of IgE responses to inhaled antigen in mice by antigen-specific γδ T cells. Science 265:1869-1871.
36. Tangri, S., L. Brossay, N. Burdin, D. J. Lee, M. Corr, and M. Kronenberg. 1998. Presentation of peptide antigens by mouse CD1 requires endosomal localization and protein antigen processing. Proc. Natl. Acad. Sci (USA) 95:14314-14319.

What is claimed is:

1. A method to inhibit airway hyperresponsiveness (AHR) in an individual, comprising deleting, inactivating or inhibiting target innate γδ T cells comprising a murine Vγ1 chain or equivalent thereof in the individual species or a Vδ5 chain or equivalent thereof in the individual species, in an individual who has or is at risk of developing AHR.

2. The method of claim 1, comprising deleting, inactivating or inhibiting target innate yd T cells comprising a murine Vγ1/Vδ5 T cell receptor, or the equivalent thereof in the individual species.

3. The method of claim 1, wherein the human equivalent is a T cell receptor comprising Vδ3.

4. The method of claim 1, wherein the human equivalent is a T cell receptor comprising Vγ9/Vδ3.

5. The method of claim 1, wherein the AHR is associated with a condition selected from the group consisting of: asthma, chronic obstructive pulmonary disease, allergic bronchopulmonary aspergillosis, hypersensitivity pneumonia, eosinophilic pneumonia, emphysema, bronchitis, allergic bronchitis bronchiectasis, cystic fibrosis, tuberculosis, hypersensitivity pneumonitis, occupational asthma, sarcoid, reactive airway disease syndrome, interstitial lung disease, hyper-eosinophilic syndrome, rhinitis, sinusitis, exercise-induced asthma, pollution-induced asthma and parasitic lung disease.

6. The method of claim 1, wherein the AHR is associated with a viral infection.

7. The method of claim 1, wherein the target innate γδ T cells are deleted, inactivated or inhibited by selective leukophoresis.

8. The method of claim 1, wherein the target innate γδ T cells are deleted, inactivated or inhibited by administration of an agent that selectively targets the γδ T cells.

9. The method of claim 1, wherein the target innate γδ T cells are deleted, inactivated or inhibited by administration of an agent that selectively targets the Vγ chain expressed by the γδ T cells.

10. The method of claim 1, wherein the target innate γδ T cells are deleted, inactivated or inhibited by administration of an agent that selectively targets the Vδ chain expressed by the γδ T cells.

11. The method of claim 8, wherein the agent is an antibody or antigen-binding fragment thereof.

12. The method of claim 11, wherein the antibody selectively targets the Vγ chain, the Vδ chain, or both the Vγ chain and the Vδ chain expressed by the γδ T cells.

13. The method of claim 8, wherein the agent is a soluble γδ T cell receptor identical or equivalent to that expressed by the γδ T cells to be deleted, inactivated or inhibited.

14. A method to inhibit airway hyperresponsiveness (AHR) in a human, comprising deleting, inactivating or inhibiting target innate γδ T cells comprising a Vδ3 chain in a human who has or is at risk of developing AHR.

15. The method of claim 14, comprising deleting, inactivating or inhibiting innate γδ T cells comprising a Vγ9/Vδ3 T cell receptor.

16. The method of claim 14, wherein the AHR is associated with a condition selected from the group consisting of: asthma, chronic obstructive pulmonary disease, allergic bronchopulmonary aspergillosis, hypersensitivity pneumonia, eosinophilic pneumonia, emphysema, bronchitis, allergic bronchitis bronchiectasis, cystic fibrosis, tuberculosis, hypersensitivity pneumonitis, occupational asthma, sarcoid, reactive airway disease syndrome, interstitial lung disease, hyper-eosinophilic syndrome, rhinitis, sinusitis, exercise-induced asthma, pollution-induced asthma and parasitic lung disease.

17. The method of claim 14, wherein the AHR is associated with a viral infection.

18. The method of claim 14, wherein the target innate γδ T cells are deleted, inactivated or inhibited by selective leukophoresis.

19. The method of claim 14, wherein the target innate γδ T cells are deleted, inactivated or inhibited by administration of an agent that selectively targets the γδ T cells.

20. The method of claim 14, wherein the target innate γδ T cells are deleted, inactivated or inhibited by administration of an agent that selectively targets the Vγ chain expressed by the γδ T cells.

21. The method of claim 14, wherein the target innate γδ T cells are deleted, inactivated or inhibited by administration of an agent that selectively targets the Vδ chain expressed by the γδ T cells.

22. The method of claim 19, wherein the agent is an antibody or antigen-binding fragment thereof.

23. The method of claim 22, wherein the antibody selectively targets the Vγ chain, the Vδ chain, or both the Vγ chain and the Vδ chain expressed by the γδ T cells.

24. The method of claim 19, wherein the agent is a soluble γδ T cell receptor identical or equivalent to that expressed by the γδ T cells to be deleted, inactivated or inhibited.
